(19) United States
(12) Patent Application Publication
(10) Pub. No.: US 2003/0088167 A1
(43) Pub. Date: May 8, 2003

(54) Biopotential electrode apparatus with integrated conductive gel switch

(75) Inventors: Charles Fendrock, Sudbury, MA (US); Shai Gozani, Brookline, MA (US)

Correspondence Address: Pandiscio & Pandiscio, 470 Totten Pond Road, Waltham, MA, 02451-1914, US

(73) Assignee: NeuroMetrix, Inc.

(21) Appl. No.: 10/036,569

(22) Filed: Nov. 7, 2001

(51) Int. Cl. A61B 5/04

(52) U.S. Cl. 600/372

(57) ABSTRACT

A biopotential electrode apparatus with an integrated conductive gel switch for easily and reliably switching interconnections on the electrode apparatus.

### FIELD OF THE INVENTION

This invention relates to apparatus and methods for monitoring electrical signals within the body, and more particularly to a biopotential electrode apparatus with an integrated conductive gel switch for selectively monitoring electrical body signals.

### BACKGROUND OF THE INVENTION

Electrodes applied to the skin are used extensively in monitoring the electrical activity of various body functions such as EKG, EEG or EMG signals.

The most widely used electrodes are generally designed to be used for a single or very specific measurement application. However, as the monitoring of biopotentials has become more complex to make more sophisticated and accurate diagnoses, so has the need for more easily configurable biopotential electrodes. Often, these biopotential electrodes are actually arrays of electrodes that can simultaneously acquire signals from multiple places on the body, or that are used to electrically stimulate neurological pathways and then measure the neurological or muscular response.

For a healthcare provider to make an assessment of a neurological, cardiac or encephalographic condition, multiple electrodes on the body are frequently required to be connected and re-connected in a combination of ways. A simple means to switch the electrode configuration can enable the healthcare provider to use one electrode array for different measurement purposes, thereby reducing costs. Sometimes this switching can be accomplished by an electronic switching means located external to the electrode or electrode array. Sometimes an elaborate or cumbersome set of wires, connectors, or harnesses mounted on the array can be employed to perform the switching. However, often because of interconnection complexity, cost or the possibility of electrical noise pick-up, it is desirable to perform the combinatorial switching through a simple, cost-effective means on the electrode itself, without the use and clumsiness of added electronic parts, connectors or jumper wires.

Many of the electrodes that are used in the medical monitoring of electrical body signals are arrays. Examples of such electrode arrays are shown and described in U.S. Pat. No. 6,032,064 issued to Devlin et al.; U.S. Pat. No. 3,490,439 issued to Rolston; U.S. Pat. No. 4,072,145 issued to Silva; U.S. Pat. No. 4,595,013 issued to Jones et al.; U.S. Pat. No. 4,638,807 issued to Ryder; and U.S. Pat. No. 6,132,387 issued to Gozani et al. These electrode arrays all provide a means to apply multiple electrodes to the body for the purpose of acquiring biological signals. However, none of the electrode arrays disclosed in these patents has the inherent capability to let the user, simply and inexpensively, electrically change the electrode configuration with an integral switching mechanism for the purpose of acquiring a different set of signals to make, for example, a more complete diagnosis.

To change the electrode configurations on some of the above-identified electrode array schemes, the only possibility is to switch connections by removing and re-attaching the wires on the individual electrodes on the array as might be possible. See, for example, the Rolston, Silva and Ryder patents. Or, one might be able to electronically select a specific set of electrodes with an external switching means.

Each of the techniques described above require either a cumbersome process of detaching and then re-attaching wires that may drape over the patient and that are often bundled together to minimize electrical noise pick-up, or require a more expensive set of switching electronics that adds complexity and cost to the signal acquisition system.

### SUMMARY OF THE INVENTION

It is, therefore, an object of the present invention to provide an integrated switching means on an electrode or electrode array which can easily and reliably be used to electrically switch interconnections on the electrode or electrode array.

Another object of the present invention is to provide an integrated switching means on an electrode or electrode array that is constructed of the same basic materials as the electrode or electrode array itself, and is manufactured with the same process steps as the electrode or electrode array, such that the construction is relatively simple and inexpensive to produce.

And another object of the present invention is to provide a biopotential electrode apparatus with an integrated conductive gel switch.

Still another object of the present invention is to provide an improved method for switching a biopotential electrode apparatus.

These and other objects are addressed by the provision and use of the present invention which, in one form of the invention, comprises an electrode or electrode array with an integrated conductive gel switch. The electrode or electrode array generally comprises a flexible substrate with a conductive pattern deposited on it by silk screening, chemical plating or other conventional means well known to those skilled in the art. The conductive pattern forms the acquisition electrode areas that acquire the biopotential signals, any stimulation electrode areas that stimulate neurological pathways within the body, and the conductive leads from the electrode areas to a periphery of the construction so that the signals may be connected to the signal monitoring electronics. A conductive gel layer resides over the acquisition electrode areas and any stimulation electrode areas and contacts and conforms to the skin. A layer of adhesive attaches the non-electrode areas to the skin. The device's integrated conductive gel switch is constructed by providing additional separate conductive patterns and conductive gel layers on the flexible substrate. These are located adjacent to the circuit patterns that need to be switched or connected in a prescribed manner so as to achieve the proper signal acquisition sequence or configuration or the proper stimulation pattern. The additional conductive gel layers are manipulated relative to the additional conductive patterns so as to effect the desired switching actions. The integrated conductive gel switch can also be used to connect or disconnect a single electrode.

In another form of the invention, there is provided a biopotential electrode apparatus for selectively monitoring the electrical activity of body functions, the biopotential electrode apparatus comprising: a substrate; a conductive interconnect pattern disposed on the substrate, the conductive interconnect pattern forming an electrode area and a conductive pathway from the electrode area to a periphery of the biopotential electrode apparatus so as to allow connection to signal monitoring electronics; a conductive gel disposed on the electrode area; and an integrated conductive gel switch for switching the electrode area between a first condition and a second condition, the integrated conductive gel switch comprising a first electrical contact electrically connected to the electrode area and a second electrical contact electrically isolated from the electrode area, and a switch conductive element selectively configurable between (i) a first state wherein the switch conductive element does not electrically connect the first electrical contact with the second electrical contact, and (ii) a second state wherein the switch conductive element electrically connects the first electrical contact with the second electrical contact, wherein the switch conductive element comprises a conductive gel.

In another form of the invention, there is provided a biopotential electrode apparatus for selectively monitoring the electrical activity of body functions, the biopotential electrode apparatus comprising: a substrate; a conductive interconnect pattern disposed on the substrate, the conductive interconnect pattern forming an electrode area and a conductive pathway from the electrode area to a periphery of the biopotential electrode apparatus so as to allow connection to signal monitoring electronics; a conductive gel disposed on the electrode area; and an integrated conductive gel switch for switching the electrode area between a first condition and a second condition, the integrated conductive gel switch comprising a first electrical contact electrically connected to the electrode area and having a layer of conductive gel thereon, and a second electrical contact electrically isolated from the electrode area and having a layer of conductive gel thereon, and a switch conductive element selectively configurable between (i) a first state wherein the switch conductive element does not electrically connect the first electrical contact with the second electrical contact, and (ii) a second state wherein the switch conductive element electrically connects the first electrical contact with the second electrical contact, wherein the switch conductive element comprises a conductor disposed on a fold-over tab attached to the substrate so that the integrated conductive gel switch assumes the second state when the fold-over tab is folded over the substrate whereby the switch conductive element is brought into electrical contact with the first and second electrical contacts, and the switch assumes the first state when the fold-over tab is not folded over the substrate.

In another form of the invention, there is provided a method for selectively monitoring electrical activity of body functions, the method comprising:

providing a biopotential electrode apparatus for selectively monitoring the electrical activity of body functions, the biopotential electrode apparatus comprising: a substrate; a conductive interconnect pattern disposed on the substrate, the conductive interconnect pattern forming an electrode area and a conductive pathway from the electrode area to a periphery of the biopotential electrode apparatus so as to allow connection to signal monitoring electronics; a conductive gel disposed on the electrode area; and an integrated conductive gel switch for switching the electrode area between a first condition and a second condition, the integrated conductive gel switch comprising a first electrical contact electrically connected to the electrode area and a second electrical contact electrically isolated from the electrode area, and a switch conductive element selectively configurable between (i) a first state wherein the switch conductive element does not electrically connect the first electrical contact with the second electrical contact, and (ii) a second state wherein the switch conductive element electrically connects the first electrical contact with the second electrical contact, wherein the switch conductive element comprises a conductive gel;

monitoring the electrode area while the integrated conductive gel switch is in the first state;

reconfiguring the integrated conductive gel switch so that the switch is in the second state; and

monitoring the electrode area while the integrated conductive gel switch is in the second state.

In another form of the invention, there is provided a method for selectively monitoring electrical activity of body functions, the method comprising:

providing a biopotential electrode apparatus for selectively monitoring the electrical activity of body functions, the biopotential electrode apparatus comprising: a substrate; a conductive interconnect pattern disposed on the substrate, the conductive interconnect pattern forming an electrode area and a conductive pathway from the electrode area to a periphery of the biopotential electrode apparatus so as to allow connection to signal monitoring electronics; a conductive gel disposed on the electrode area; and an integrated conductive gel switch for switching the electrode area between a first condition and a second condition, the integrated conductive gel switch comprising a first electrical contact electrically connected to the electrode area and having a layer of conductive gel thereon, and a second electrical contact electrically isolated from the electrode area and having a layer of conductive gel thereon, and a switch conductive element selectively configurable between (i) a first state wherein the switch conductive element does not electrically connect the first electrical contact with the second electrical contact, and (ii) a second state wherein the switch conductive element electrically connects the first electrical contact with the second electrical contact, wherein the switch conductive element comprises a conductor disposed on a fold-over tab attached to the substrate so that the integrated conductive gel switch assumes the second state when the fold-over tab is folded over the substrate whereby the switch conductive element is brought into electrical contact with the first and second electrical contacts, and the switch assumes the first state when the fold-over tab is not folded over the substrate;

monitoring the electrode area while the integrated conductive gel switch is in the first state;

reconfiguring the integrated conductive gel switch so that the switch is in the second state; and

monitoring the electrode area while the integrated conductive gel switch is in the second state.

### BRIEF DESCRIPTION OF THE DRAWINGS

These and other objects and features of the present invention will be more fully disclosed or rendered obvious by the following detailed description of the preferred embodiments of the invention, which are to be considered together with the accompanying drawings wherein like numbers refer to like parts, and further wherein:

FIG. 1 is a schematic perspective view of one preferred embodiment of the present invention, showing an electrode array with an integrated fold-over conductive gel switch tab;

FIG. 2 is a schematic cross-sectional view of the electrode array shown in FIG. 1, taken along line 2-2 of FIG. 1;

FIG. 3 is a schematic plan view of another preferred embodiment of the present invention, showing an alternate implementation of an integrated fold-over conductive gel switch tab;

FIG. 4 is a schematic perspective view of another preferred embodiment of the present invention, showing a pair of integrated fold-over conductive gel switch tabs;

FIG. 5 is a schematic perspective view of another preferred embodiment of the present invention, showing an integrated conductive gel switch having a solid switch conductor on a fold-over tab and conductive gel layers on the two electrical contacts on the substrate;

FIG. 6 is a schematic perspective view of another preferred embodiment of the present invention, showing an integrated conductive gel switch having a flexible dome with conductive gel movably disposed therein;

FIG. 7 is a schematic cross-sectional view of the integrated conductive gel switch shown in FIG. 6, taken along line 7-7 of FIG. 6;

FIG. 8 is a schematic top view of another preferred embodiment of the present invention, showing an integrated conductive gel switch comprising a recess covered by a non-conductive liner, and a conductive gel movably disposed therein, whereby to create a concave conductive gel switch; and

FIG. 9 is a schematic cross-sectional view of the integrated conductive gel switch of FIG. 8, taken along line 9-9 of FIG. 8.

### DETAILED DESCRIPTION OF THE PREFERRED EMBODIMENTS

The preferred embodiments of the present invention, including various novel details of construction and combinations of parts and method steps, will now be more particularly described with reference to the accompanying drawings. It will be understood that the particular devices and method steps embodying the invention are shown by way of illustration only and not as limitations of the invention. The principles and features of this invention may be employed in various and numerous embodiments without departing from the scope of the invention.

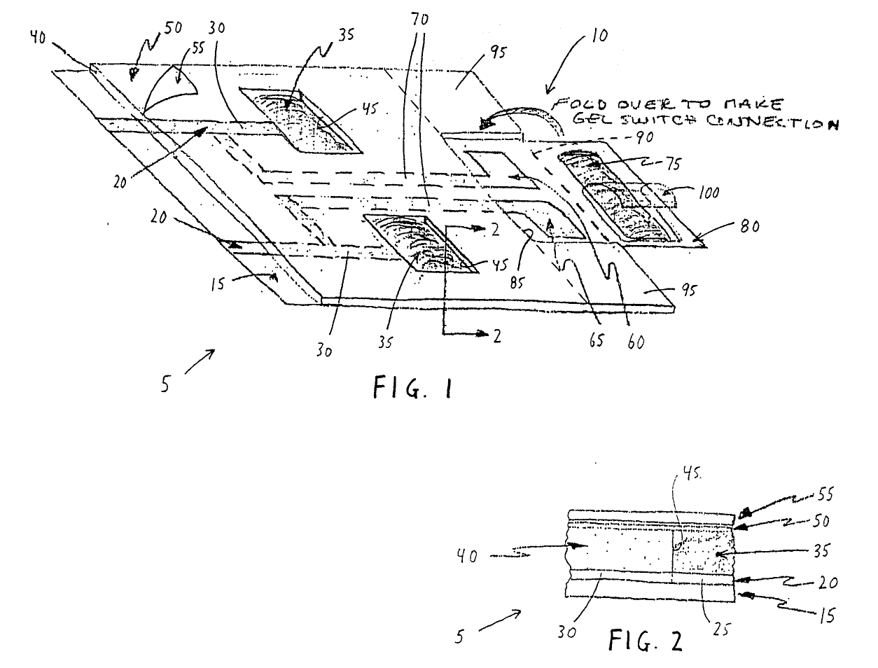

Looking now at FIGS. 1 and 2, there is shown a biopotential electrode apparatus 5 which includes an integrated conductive gel switch 10.

Biopotential electrode apparatus 5 comprises a substrate 15. Substrate 15 is preferably flexible, although it may also be substantially rigid if desired. In one preferred form of the invention, substrate 15 is formed out of mylar or a similar material.

A conductive interconnect pattern 20 is disposed on substrate 10 and includes electrode areas 25 (FIG. 2) and conductive lead connections 30 (FIGS. 1 and 2) for connection to signal monitoring electronics (not shown). In one form of the invention, conductive interconnect pattern 20 is formed by applying conductive ink to the upper surface of substrate 15.

A layer of conductive gel 35 (FIGS. 1 and 2) is placed over electrode areas 25. Conductive gel 35 may be any one of the many conductive gels commercially available, and preferably comprises a conductive gel which is initially deployed on electrode areas 25 in liquid form and then UV-cured in situ. Such a conductive gel is commercially available from the Promeon division of Medtronic of Minneapolis, Minn. Alternatively, conductive gel 35 may comprise a non-UV-cured hydrogel such a liquid hyrdogel that has a viscous consistency. To the extent that conductive gel 35 is initially deployed on electrode areas 25 in liquid form, conductive gel 35 is preferably initially maintained over electrode areas 25 by a foam tape mask 40. More particularly, foam tape mask 40 is disposed on substrate 15 and includes windows 45 (FIGS. 1 and 2) located over electrode areas 25. Windows 45 act as a reservoir to hold the liquid conductive gel 35 centered over electrode areas 25 until the conductive gel is cured in situ. In one preferred embodiment of the present invention, foam tape mask 40 has a thickness between about 0.005 and 0.080 inches, and conductive gel 35 has a thickness between about 0.010 and about 0.100 inches.

Biopotential electrode apparatus 5 also comprises an adhesive 50 applied to the upper surface of tape 40 for maintaining the device against the skin of a patient, and a protective release liner 55 applied to adhesive 50 and removable at the time of use.

Integrated conductive gel switch 10 comprises a first electrical contact 60 and a second electrical contact 65. First electrical contact 60 and second electrical contact 65 are electrically connected to electrode areas 25 (e.g., by the aforementioned conductive interconnect pattern 20 and an additional conductive interconnect pattern 70) but normally electrically isolated from one another. Integrated conductive gel switch 10 also comprises a layer of conductive gel 75 disposed on a switch tab 80. Switch tab 80 is adapted to be folded over first electrical contact 60 and second electrical contact 65 so as to bring conductive gel 75 into contact with the same, whereby to electrically connect first electrical contact 60 with second electrical contact 65 and thereby change the state of integrated conductive gel switch 10. In order to facilitate folding over switch tab 80 so as to establish the aforementioned electrical contact, switch tab 81 may constitute an extension of substrate 15, and foam tape 40 may be cut back as at 85 in the region of electrical contacts 60 and 65. Substrate 15 may also be pre-creased such as shown at 90 so as to facilitate folding. In addition, adhesive 50 may be omitted from the upper surface of foam tape 40 in the region 95 so as to facilitate lifting the switch end of the device away from the patient's skin, so as to enable switch tab 80 to be easily folded over against electrical contacts 60 and 65. A protective release liner 100 normally overlies the switch's conductive gel 75.

In use, biopotential electrode apparatus 5 has its protective release liner 55 removed and the apparatus is applied, adhesive layer 50 first, to the skin of the patient. At this point integral conductive gel switch 10 is in its first, open state, with switch tab 80 retracted away from first electrical contact 60 and second electrical contact 65 so that the two electrical contacts are electrically isolated from one another. When it is determined that integrated conductive gel switch 10 should change states, first electrical contact 60 and second electrical contact 65 are connected to one another, i.e., by peeling away the switch's protective release liner 100 and folding switch tab 80 back over substrate 15 so that the switch's conductive gel 75 comes in contact with first electrical contact 60 and second electrical contact 65 and forms an electrical connection therebetween, whereby to place integrated conductive gel switch 10 in its second, closed state. Returning switch tab 80 to its original position reopens the switch. Conductive gel 75 thus effectively makes an electrical connection between first electrical contact 60 and second electrical contact 65 in a Single Pole Single Throw (SPST) switching action.

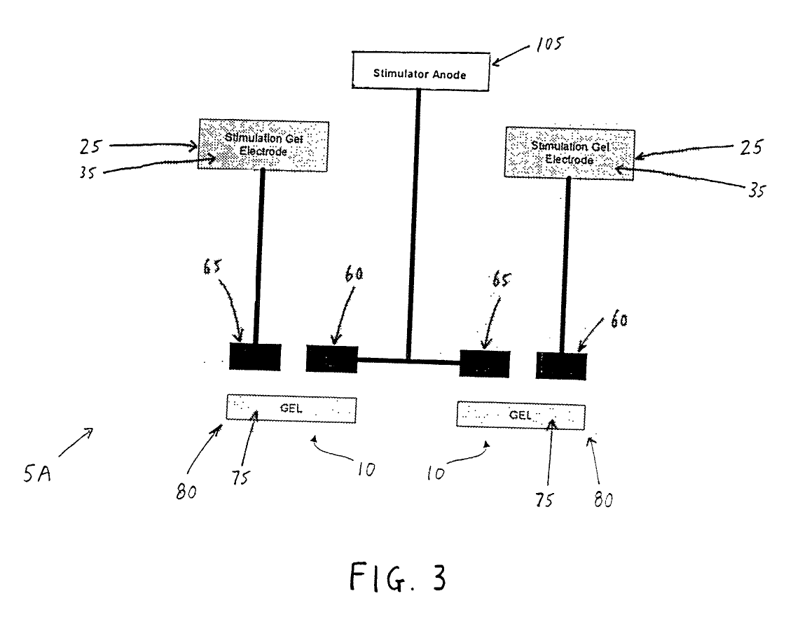

Referring now to FIG. 3, in another preferred embodiment of the present invention, a biopotential electrode apparatus 5A includes a pair of integrated conductive gel switches 10 that may be used to selectively connect or disconnect a stimulator anode 105 to stimulation electrode areas 25. Again, switches 10 are normally in their first, open state but may be placed in their second, closed state by folding switch tab 80 back over the body of the construct, whereby to cause conductive gel 75 to come into contact with first electrical contact 60 and second electrical contact 65 and thus form an electrical connection therebetween.

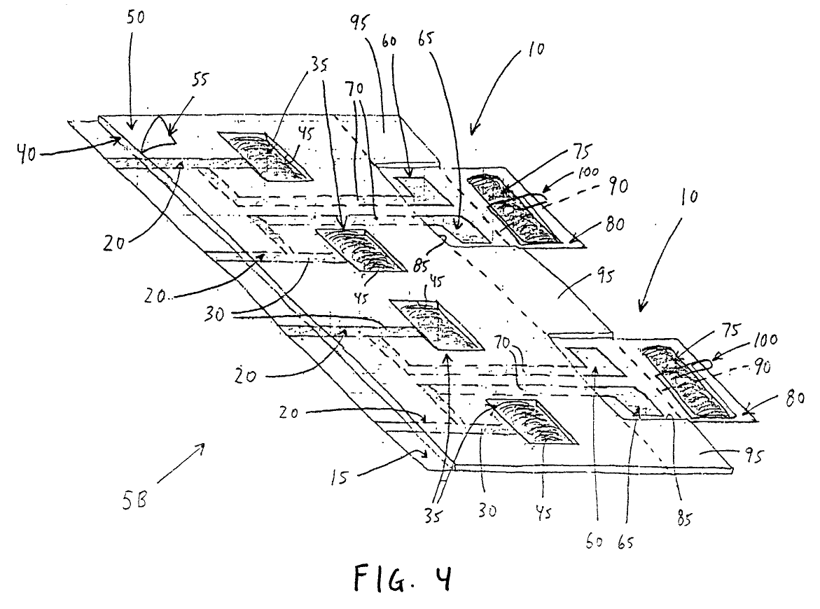

Referring now to FIG. 4, in another preferred embodiment of the present invention, a biopotential electrode apparatus 5B is shown having a plurality of integrated conductive gel switches 10. These integrated conductive gel switches 10 may be used to perform multiple connects or disconnects using the same steps as described above. The integrated conductive gel switches 10 may also be used to construct a biopotential electrode apparatus with a multitude of switching configurations commonly known as, but not limited to, Single Pole Double Throw (SPDT), Double Pole Single Throw (DPST) and the like.

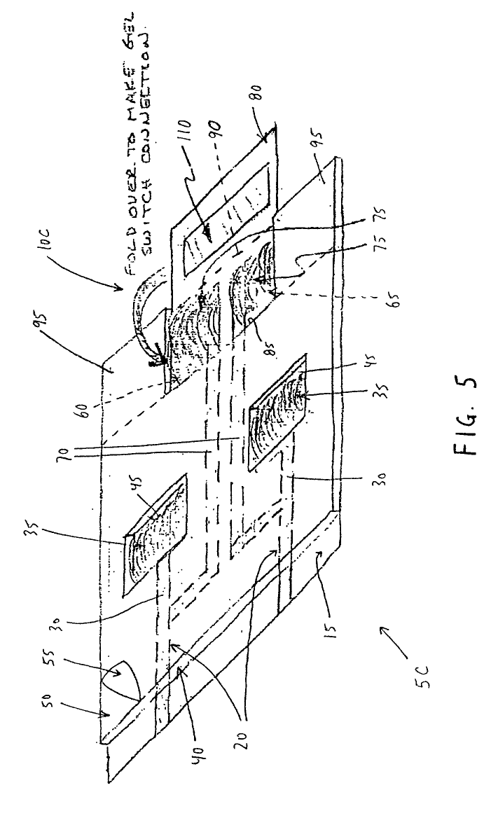

Referring now to FIG. 5, in another preferred embodiment of the present invention, a biopotential electrode apparatus 5C includes an integrated conductive gel switch 10C. Integrated conductive gel switch 10C has a conductor 110 mounted to its switch tab 80, a layer of conductive gel 75 disposed on its first electrical contact 60 and a layer of conductive gel 75 disposed on its second electrical contact 65. A protective release liner (not shown) preferably overlies the switch's two layers of conductive gel 75. Conductor 110 and the two layers of conductive gel 75 form the switch by connection and disconnection from one another. More particularly, integrated conductive gel switch 10C is in its first, open state when switch tab 80 is withdrawn from the two layers of conductive gel 75. Switch 10C is placed in its second, closed state by peeling away the protective release liner (not shown) disposed over the two layers of conductive gel 75 and then folding switch tab 80 over substrate 15 so that conductor 110 comes into contact with the two layers of conductive gel 75, whereby the two layers of conductive gel 75 (and hence electrical contacts 60 and 65) will be electrically connected to one another. The integrated conductive gel switch 10C may thereafter be reopened simply by unfolding switch tab 80 so that conductor 110 no longer electrically connects the two layers of conductive gel 75. In a similar manner, single or multiple pole, and single or multiple throw, switch configurations may be constructed using this novel construction.

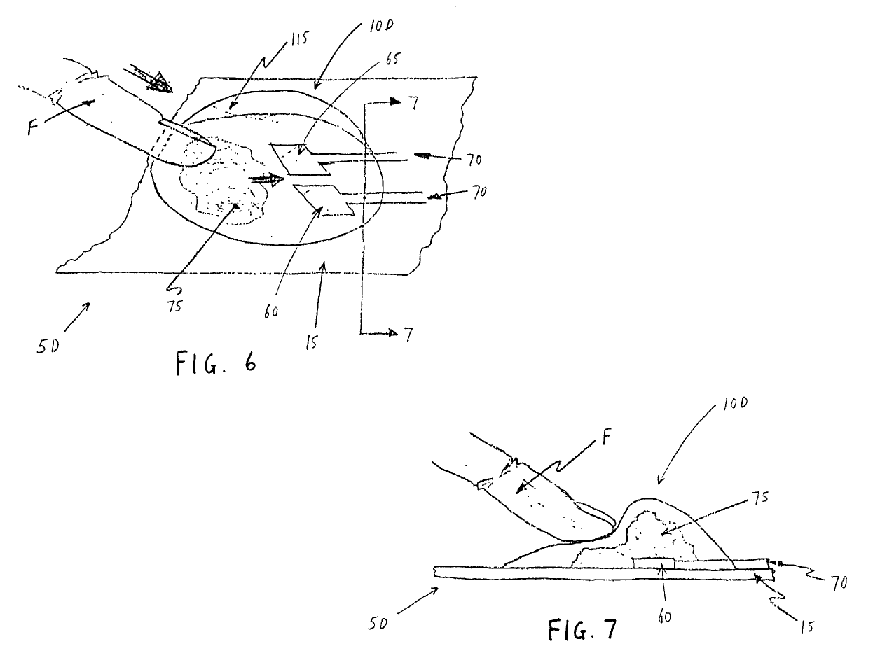

Referring now to FIGS. 6 and 7, in another preferred embodiment of the present invention, biopotential electrode apparatus 5D comprises an integrated conductive gel switch 10D. Switch 10D comprises a dome 115 formed from a flexible material which rises up over the first electrical contact 60 and the second electrical contact 65 which are to be connected to or disconnected from one another. Of course, more than two contacts 60, 65 may also be provided if desired. A mass of conductive gel 75 is movably disposed within dome 115. Pushing on one side of dome 115 with a finger F or other implement causes the mass of conductive gel 75 to move toward the other side of dome 115. In one direction this action causes the mass of conductive gel 75 to overlie, and thereby electrically connect, the two electrical contacts 60, 65 that lie under the dome. In the other direction this action displaces the mass of conductive gel from the two electrical contacts 60, 65 and thereby electrically disconnects the two contacts 60, 65 from one another. In a similar manner as described above, multiple switching configurations may be constructed from the basic domed switch 5D.

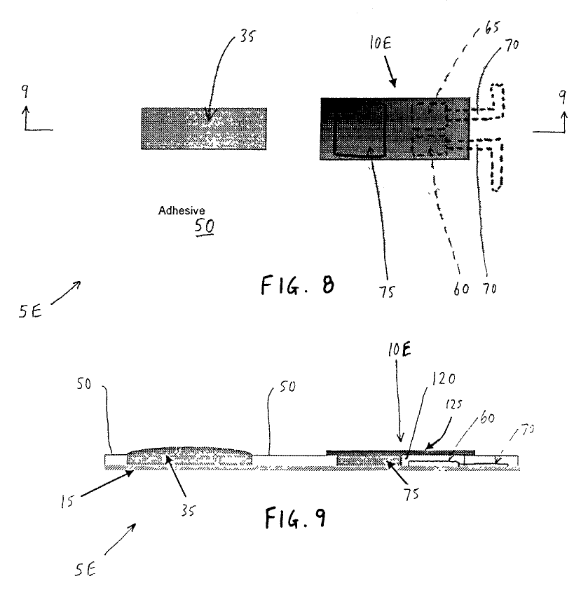

Referring now to FIGS. 8 and 9, in another preferred embodiment of the present invention, biopotential electrode apparatus 5E includes an integrated conductive gel switch 10E. Switch 10E comprises a recess 120 formed in the construction. The two electrical contacts 60, 65 are disposed in one portion of recess 120. A mass of conductive gel 75 is disposed in another portion of the recess. A flexible membrane 125 overlies recess 120. This construction effectively creates a flat version of the domed switch 5D. When the flexible membrane 120 is appropriately pushed, e.g., with a finger or other implement, the mass of conductive gel 75 may be moved laterally so as to overlie, and thereby electrically connect, the two electrical contacts 60, 65 so as to close the switch.

The apparatus and methods of the present invention utilize simple and inexpensive construction techniques commonly used in manufacturing electrodes for monitoring the electrical activity of body functions.

While the foregoing invention has been described with reference to its preferred embodiments, various alterations and modifications will occur to those skilled in the art in view of the present disclosure. All such alterations and modifications are considered to fall within the scope of the present invention.

What is claimed is:

1. A biopotential electrode apparatus for selectively monitoring the electrical activity of body functions, the biopotential electrode apparatus comprising:
a substrate;
a conductive interconnect pattern disposed on said substrate, said conductive interconnect pattern forming an electrode area and a conductive pathway from said electrode area to a periphery of said biopotential electrode apparatus so as to allow connection to signal monitoring electronics;
a conductive gel disposed on said electrode area; and
an integrated conductive gel switch for switching said electrode area between a first condition and a second condition, said integrated conductive gel switch comprising a first electrical contact electrically connected to said electrode area and a second electrical contact electrically isolated from said electrode area, and a switch conductive element selectively configurable between (i) a first state wherein said switch conductive element does not electrically connect said first electrical contact with said second electrical contact, and (ii) a second state wherein said switch conductive element electrically connects said first electrical contact with said second electrical contact, wherein said switch conductive element comprises a conductive gel.

2. A biopotential electrode apparatus according to claim 1 wherein said substrate is flexible.

3. A biopotential electrode apparatus according to claim 1 wherein said substrate comprises mylar.

4. A biopotential electrode apparatus according to claim 1 wherein said conductive interconnect pattern is disposed on a surface of said substrate material.

5. A biopotential electrode apparatus according to claim 1 wherein said conductive interconnect pattern comprises conductive ink.

6. A biopotential electrode apparatus according to claim 1 further comprising means for maintaining said conductive gel disposed on said electrode area.

7. A biopotential electrode apparatus according to claim 6 wherein said means for maintaining comprise tape disposed on said substrate and a portion of said conductive interconnect pattern.

8. A biopotential electrode apparatus according to claim 7 wherein said tape includes an opening therein, said opening being disposed about said electrode area, and said conductive gel being disposed in said opening.

9. A biopotential electrode apparatus according to claim 7 wherein said tape has an adhesive disposed thereon.

10. A biopotential electrode apparatus according to claim 1 wherein said switch conductive element is disposed on a fold-over tab attached to said substrate so that said integrated conductive gel switch assumes said second state when said fold-over tab is folded over said substrate whereby said switch conductive element is brought into electrical contact with said first and second electrical contacts, and said switch assumes said first state when said fold-over tab is not folded over said substrate.

11. A biopotential electrode apparatus according to claim 10 wherein said fold-over tab is formed integral with said substrate.

12. A biopotential electrode apparatus according to claim 10 wherein said switch conductive element comprises a layer of conductive gel fixed to said fold-over tab.

13. A biopotential electrode apparatus according to claim 12 wherein said layer of conductive gel has a protective release liner disposed thereon.

14. A biopotential electrode according to claim 1 wherein said switch conductive element comprises a mass of conductive gel movable relative to said first and second electrical contacts.

15. A biopotential electrode apparatus according to claim 14 wherein said mass of conductive gel is located within a flexible dome structure.

16. A biopotential electrode apparatus according to claim 14 wherein said mass of conductive gel is located within a recess and covered by a flexible membrane.

17. A biopotential electrode apparatus according to claim 1 wherein said integrated conductive gel switch includes the same type of material as said substrate and the same type of material as said conductive gel disposed on said electrode area.

18. A biopotential electrode apparatus according to claim 1 further comprising an additional electrode area, wherein said electrode area comprises a biopotential signal acquiring electrode and said additional electrode area comprises a stimulation electrode.

19. A biopotential electrode apparatus for selectively monitoring the electrical activity of body functions, said biopotential electrode apparatus comprising:
a substrate;
a conductive interconnect pattern disposed on said substrate, said conductive interconnect pattern forming an electrode area and a conductive pathway from said electrode area to a periphery of said biopotential electrode apparatus so as to allow connection to signal monitoring electronics;
a conductive gel disposed on said electrode area; and
an integrated conductive gel switch for switching said electrode area between a first condition and a second condition, said integrated conductive gel switch comprising a first electrical contact electrically connected to said electrode area and having a layer of conductive gel thereon, and a second electrical contact electrically isolated from said electrode area and having a layer of conductive gel thereon, and a switch conductive element selectively configurable between (i) a first state wherein said switch conductive element does not electrically connect said first electrical contact with said second electrical contact, and (ii) a second state wherein said switch conductive element electrically connects said first electrical contact with said second electrical contact, wherein said switch conductive element comprises a conductor disposed on a fold-over tab attached to said substrate so that said integrated conductive gel switch assumes said second state when said fold-over tab is folded over said substrate whereby said switch conductive element is brought into electrical contact with said first and second electrical contacts, and said switch assumes said first state when said fold-over tab is not folded over said substrate.

20. A method for selectively monitoring electrical activity of body functions, said method comprising:
providing a biopotential electrode apparatus for selectively monitoring the electrical activity of body functions, said biopotential electrode apparatus comprising:
a substrate;
a conductive interconnect pattern disposed on said substrate, said conductive interconnect pattern forming an electrode area and a conductive pathway from said electrode area to a periphery of said biopotential electrode apparatus so as to allow connection to signal monitoring electronics;
a conductive gel disposed on said electrode area; and
an integrated conductive gel switch for switching said electrode area between a first condition and a second condition, said integrated conductive gel switch comprising a first electrical contact electrically connected to said electrode area and a second electrical contact electrically isolated from said electrode area, and a switch conductive element selectively configurable between (i) a first state wherein said switch conductive element does not electrically connect said first electrical contact with said second electrical contact, and (ii) a second state wherein said switch conductive element electrically connects said first electrical contact with said second electrical contact, wherein said switch conductive element comprises a conductive gel;
monitoring said electrode area while said integrated conductive gel switch is in said first state;
reconfiguring said integrated conductive gel switch so that said switch is in said second state; and
monitoring said electrode area while said integrated conductive gel switch is in said second state.

21. A method for selectively monitoring electrical activity of body functions, said method comprising:
providing a biopotential electrode apparatus for selectively monitoring the electrical activity of body functions, said biopotential electrode apparatus comprising:
a substrate;
a conductive interconnect pattern disposed on said substrate, said conductive interconnect pattern forming an electrode area and a conductive pathway from said electrode area to a periphery of said biopotential electrode apparatus so as to allow connection to signal monitoring electronics;
a conductive gel disposed on said electrode area; and
an integrated conductive gel switch for switching said electrode area between a first condition and a second condition, said integrated conductive gel switch comprising a first electrical contact electrically connected to said electrode area and having a layer of conductive gel thereon, and a second electrical contact electrically isolated from said electrode area and having a layer of conductive gel thereon, and a switch conductive element selectively configurable between (i) a first state wherein said switch conductive element does not electrically connect said first electrical contact with said second electrical contact, and (ii) a second state wherein said switch conductive element electrically connects said first electrical contact with said second electrical contact, wherein said switch conductive element comprises a conductor disposed on a fold-over tab attached to said substrate so that said integrated conductive gel switch assumes said second state when said fold-over tab is folded over said substrate whereby said switch conductive element is brought into electrical contact with said first and second electrical contacts, and said switch assumes said first state when said fold-over tab is not folded over said substrate;
monitoring said electrode area while said integrated conductive gel switch is in said first state;
reconfiguring said integrated conductive gel switch so that said switch is in said second state; and
monitoring said electrode area while said integrated conductive gel switch is in said second state.
